(19) United States
(12) Patent Application Publication
(10) Pub. No.: US 2020/0043099 A1
(43) Pub. Date: Feb. 6, 2020

(54) HEALTH INSURANCE CLAIM CODING SYSTEM

(71) Applicants: Vatsal Girish Thakkar, New York, NY (US); William L. Brown, New York, NY (US)

(72) Inventors: Vatsal Girish Thakkar, New York, NY (US); William L. Brown, New York, NY (US)

(21) Appl. No.: 16/527,156

(22) Filed: Jul. 31, 2019

Related U.S. Application Data
(60) Provisional application No. 62/712,303, filed on Jul. 31, 2018.

(51) Int. Cl. G06Q 40/08; G16H 10/60; G16H 15/00; G06K 19/06

(52) U.S. Cl. CPC G06Q 40/08; G06K 19/06037; G16H 15/00; G16H 10/60

(57) ABSTRACT

A rapid and high-fidelity health insurance coding system comprising a network and a database in communication with the network to store patient data and medical practitioner data is provided. A provider terminal is utilized by the medical practitioner to input a plurality of codes, each corresponding to a patient visit. A health insurance claim processing engine receives the codes and automatically generates a comprehensive and often complete health insurance claim form from at least one of the plurality of codes. The health insurance claim form is then transmitted via the network to an insurance payer.

### CROSS-REFERENCE TO RELATED APPLICATIONS

The present application claims priority to U.S. Provisional Application No. 62/712,303 filed on Jul. 31, 2018, entitled “HEALTH INSURANCE CLAIM CODING SYSTEM” the entire disclosure of which is incorporated by reference herein.

### TECHNICAL FIELD

The embodiments generally relate to a specialized coding system to improve efficiency and reduce errors in health insurance claims.

### BACKGROUND

The CMS-1500 is the standard health insurance claim form and is the foundation upon which all health insurance claims for both public and private insurance plans are based. The current CMS-1500, OMB #0938-1197, is a one-page document that consists of thirty-three item categories that comprise 232 individual fields of data entry. The electronic version of this claim form is the 837 Professional (837P) and is considered equivalent to and synonymous with the CMS-1500.

The CMS-1500 brings together data elements from at least three separate health care entities involved in the transaction for which the claim is being submitted. These three entities are the health insurance payer, the healthcare service provider/vendor, and the transaction's consumer, typically identified as the patient.

The basic provider and practice/organization information, which typically remains the same for all healthcare claims for that provider/organization, corresponds to the following fields on the CMS-1500: 24B, 24J, 25, 27,32, 32a, 33, 33a.

The healthcare service provider/vendor specifies further data elements necessary for the CMS-1500 in the form of the International Classification of Diseases, 10th Revision codes (abbreviated as “ICD-10” codes, also known as “diagnosis” or “dx” codes) and Current Procedural Terminology codes (abbreviated as “CPT” codes, also known as “procedure” or “service” codes), and the fees for each of the CPT codes, along with other bits of data. These data elements do not vary significantly for any given provider or entity, based on their specialty. For example, in his/her career, a physician may only utilize 30-40 diagnosis codes (of the tens of thousands available), 5-10 CPT codes (of the thousands available), and may only have five different fees based on his/her practice type and setting. The corresponding fields on the CMS-1500 claim form are 21, 24D, 24E, 24F, and 24G.

The final bits of data are provided by the patient and the primary insured for the health insurance policy. This data includes the names, demographic information (address, date of birth, etc.), along with the type of insurance policy, ID number, etc. The corresponding fields on the CMS-1500 are fields 1, 2, 3, 4, 5, 6, 7, 8, 9, 10, 11, 12, and 13 (all fields between 1-13).

The current NPI (National Provider Identifier) system, which is designed, executed, and maintained by the United States Department of Health and Human Services is limited for purposes of providing a ten-digit code for health insurance claim processing. While the NPI database does identify almost all of the millions of individual health care providers as well as the millions of healthcare organizations in the United States, it does not accomplish the following, which are necessary features to complete a health insurance claim. First, the NPI database does not make available a provider's or organization's Taxpayer Identification Number (or Employer Identification Number, EIN). Further, the NPI database does not link individual practitioners and the NPI numbers with practices, hospitals, and/or healthcare organizations which have their own, distinct NPI numbers (defined in the NPI system as a Type II NPI number). The NPI information for both the provider and the organization needs to be included on a health insurance claim. Also, the NPI database identifies the provider's specialty (defined as “taxonomy”), but does not offer a compendium of the providers more frequently used ICD-10 codes, CPT codes, or fees.

There does not exist today a systematic, streamlined means of clustering these claim data elements together for repeated use for the purposes of enhanced efficiency, faster claim filing and processing, error reduction, and facilitation for any party in the healthcare transaction to initiate a health insurance claim.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments disclosed herein provide a rapid and high-fidelity health insurance coding system comprising a network and a database in communication with the network to store patient data and medical practitioner data. A provider terminal is utilized by the medical practitioner to input a plurality of codes, each corresponding to a patient visit. A medical insurance claim processing engine receives the codes and automatically generates a medical insurance claim form from at least one of the plurality of codes. The medical insurance claim form is then transmitted via the network to an insurance payer. The embodiments provide a systematic and streamlined means for gathering claim data elements for repeated use. This system enhances efficiency, reduces errors, and facilitates communication between multiple healthcare parties.

In one aspect, the medical insurance claim processing engine receives at least one of the following: a service package name, one or more diagnostic codes, one or more CPT codes, and one or more fees for any given health care provider or facility.

In one aspect, the fee is associated with the service package name.

In one aspect, the medical practitioner or system administrator inputs a plurality of service package names each stored in the database.

In one aspect, the medical practitioner or system administrator inputs a plurality of diagnostic codes, each corresponding to a patient diagnosis. The plurality of diagnostic codes is stored in the database.

In one aspect, the code is customizable via the medical provider or system administrator to input codes most relevant to the provider's area of medical practice.

In one aspect, the network is accessible by one or more insurance payers, health care vendors, or other parties involved in the health care transaction.

In one embodiment, the code can be auto-generated based on actual health insurance claims being filed in real-time on the network.

In some embodiments, a method for generating a health insurance claim form is disclosed comprising the steps of inputting, via a medical practitioner utilizing a provider terminal, a plurality of codes corresponding to patient information. Next, an interpretation module interprets the plurality of codes which are then transmitted to a medical insurance claim processing engine which automatically generates a medical insurance claim form using the plurality of codes. The medical insurance claim form is then transmitted to an insurance payer.

### BRIEF DESCRIPTION OF THE DRAWINGS

A more complete understanding of the present embodiments and the advantages and features thereof will be more readily understood by reference to the following detailed description when considered in conjunction with the accompanying drawings wherein:

FIG. 1 illustrates a block diagram of a medical insurance claim terminal, according to some embodiments;

FIG. 2 illustrates a screenshot of the medical insurance code creation/editing interface, according to some embodiments;

FIG. 3 illustrates a server engine configuration, according to some embodiments;

FIG. 4 illustrates a Quick Response (QR) code generated from the unique code generated by the system, according to some embodiments; and

FIG. 5 illustrates a flowchart for a method for automatically generating a medical insurance claim form, according to some embodiments.

### DETAILED DESCRIPTION

The specific details of the single embodiment or variety of embodiments described herein are to the described system. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

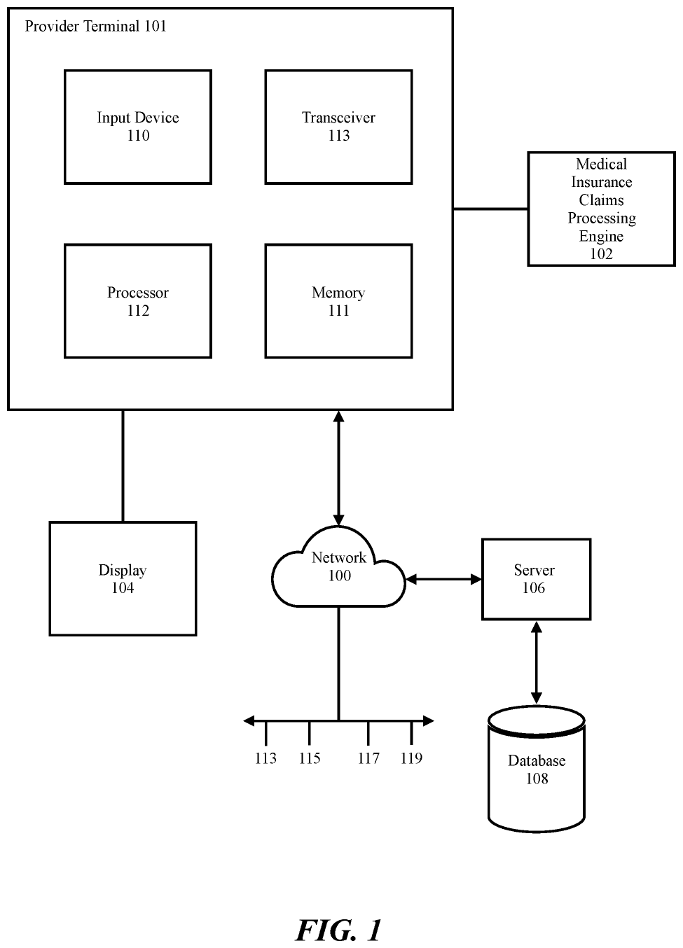

FIG. 1 depicts a preferred embodiment of a healthcare provider terminal 101 such as those which could be found in a doctor's office, health clinic, hospital, dental office, central system administrator, or any other place in which health care services are rendered to patients. The terminal 101 according to the present embodiments include a keyboard, computerized dictation system, or other wired or wireless data input device in communication with the memory 111 and which may be used to input medical insurance claim data into the terminal 101. Medical insurance claim data entered via the input device 110 is stored in the memory 111, which may be any data storage device, such as a hard drive, CD-ROM, DVD, floppy disk, flash memory, or other data storage device as would be apparent to one of skill in the art. The terminal includes a display 104 permitting engagement with a user interface thereon.

The provider terminal 101 also includes a processor 112 that receives medical insurance claim data from the input device 110 that facilitates the storage of the data in the memory 111 and processes the data and other data as described below. Medical insurance claim data may be transmitted via a transceiver for transmitting and receiving data. The transceiver may be any transceiver for sending and receiving data, such as a modem coupled to a telephone line, broadband connection, satellite connection, Internet connection, or cable connection, or any other wired or wireless data communication network as would be apparent to one of skill in the art. The transceiver receives medical insurance claim data from processor 112 and transmits it to a medical insurance claim processor 102, which generates the medical insurance claim and associated claim form (either the CMS-1500 or its electronic equivalent the 837P) from input received from the provider terminal 101.

Data input device 110 is configured to prepare and store a generated code. The processor 112 includes processing instructions to establish a code consisting of a plurality of alphanumeric characters that encode substantially all of the data elements needed for a single health insurance claim.

The code can be generated and used in several ways. In one example, a provider can register their various practice settings along with their most commonly used ICD codes, CPT codes, and fees based on their area of specialty. This will enumerate various codes for the provider's use. In another example, a practice or medical organization can register each provider who provides services through that practice or organization. In yet another example, a centralized claims processor such as a health insurance claims clearinghouse or a third-party billing organization may come across these common data elements for individual providers or organizations and wish to establish the code set for the provider or organization. In another example, a patient may be given the code to use for filing their own health insurance claims.

The codes will reside in a database 108 that can be accessible to various parties in the health insurance claim and healthcare industries. Because codes are unique identifiers, they cannot be duplicated and therefore must be verified and validated against a centralized registry.

A particular code may be sequentially structured such that the initial characters will be fixed to represent those parts of the health insurance claims that are least likely to vary on a regular basis. The latter characters in the code may become more specific in some embodiments. In addition, one practitioner may have several codes as follows to represent both different practice settings as well as various combinations of services and fees.

In one embodiment, the logic comprises software stored on the input device 110. In another embodiment, the logic comprises software stored on the database 108 accessible via server 106 with which the device can communicate over a network 100. The network can include each party involved in the medical insurance claim. These parties include the patient 113, the care provider 115, and the insurance payer 117. In another embodiment, at least one additional user 119 can be in communication with the network 100 throughout the process of filing the claim.

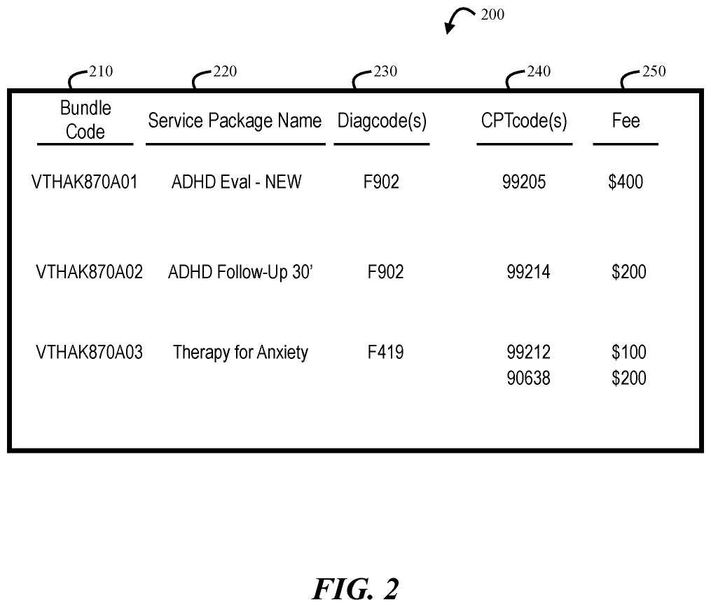

In reference to FIG. 2, an exemplary screenshot of the medical insurance claim processing interface 200 is illustrated comprising a code column 210, an exam name column 220, a diagnostic code(s) column 230, a CPT code(s) column 240, and a fee column 250. The code column 210 includes a plurality of automatically generated codes which are utilized to automatically fill an insurance claim form which corresponds to a patient's visit to a medical practitioner. The exam name column 220 is comprised of exam descriptions or exam codes input by the medical practitioner following a patient and practitioner interaction, such as, for example, a new patient evaluation for attention-deficit/hyperactivity disorder (ADHD). The diagnostic code(s) column 230 is comprised of codes input by the medical practitioner, which relate to the exam description and the eventual diagnosis of the patient. The CPT code(s) column 240 is comprised of CPT codes as described herein while the fee column 250 indicates the fee total for each CPT code as well as the total for that combined service package.

Table 1 below illustrates an exemplary embodiment of a code generation scheme. It is understood that variations to this scheme can be employed by the processor 112. For example, a case-sensitive scheme can be utilized. In another embodiment, numeric-only and/or alpha-only character schemes can be used. In yet another embodiment, instead of the first initial and 3 letters of the provider's last name, 2 from the first and 2 from the last could be used (see section A in Table 1 below).

TABLE 1
Exemplary Code Scheme

| | | |
|---|---|---|
| A | RLOW | Base of practitioner name: first letter of the first name followed |
| | | by a minimum of 3 letters of the last name. More letters can be |
| | | used to distinguish from other HICCS codes. If the last name has |
| | | fewer than 3 letters, then the name base (SHICCS-A) can be fewer |
| | | than 4 letters. (For example for a practitioner named John Le, the |
| | | name base can be “JLE”) |
| B | 987 | Base of Final digits of the practitioners NPI number: Typically |
| | | the final 3 digits will be used. If this causes a duplicate HICCS code |
| | | with another practitioner, then the final 4 digits of the NPI number |
| | | will be used, to ensure a unique HICCS code for every practitioner. |
| C | A | Practice/Organization Specifier: This indicates a unique |
| | | practice setting for this provider. This specifier includes data for |
| | | all of the following health insurance claim fields: |
| | | Place of Service (24B) |
| | | Service Provider Federal Tax ID Number (25) |
| | | Service Facility Location (32) |
| | | Service Facility NPI Number (32A), if different from |
| | | Provider NPI number |
| | | Billing Provider Info (33) |
| | | Billing Provider NPI Number (33A), if different from |
| | | Provider NPI number |
| D | A6D4C | Clinical Bundle Code (CBC): the core triad of a health insurance |
| | D1: A6 | claim requires: diagnosis code(s), in ICD-10 format, procedure |
| | D2: D4 | code(s), in CPT code format, and the fee for each CPT code listed. |
| | D3: C | The clinical bundle code of the SHICCS is defined as follows: |
| | | D1--the first letter defines the CPT/HCPCS code(s) plus |
| | | modifiers (24D): |
| | | if there are more than 26 bundles (comprising |
| | | letters A-Z), then numbering continues with A1, B1, |
| | | C1, etc. for the next 26 bundles, after which |
| | | numbering continues as A2, B2, C2, etc. |
| | | D2--the 2nd letter defines the associated fee (24F) for |
| | | each CPT code in this clinical bundle. Numbering past the |
| | | first 26 bundles continues as described in the manner |
| | | described above for the 1^{st} letter of the CBC. |
| | | D3--the 3^{rd} letter defines the diagnosis code(s) (21, |
| | | 24E): |
| | | A minimum of one |
| | | Numbering past the first 26 bundles (comprising |
| | | letters A-Z) continues as described above for the |
| | | other letters of the CBC. |
| | | The sequence of these three letters is in hierarchical order: |
| | | While many CBCs will indicate the entire claim triad |
| | | of ICD-10 codes, CPT/HCPCS codes, and fees, a CBC |
| | | can be created, which is not a complete triad. |
| | | If a CBC consists of one letter, it indicates only one or |
| | | more CPT codes for the claim triad, and therefore |
| | | the fee(s) and diagnosis/ICD-10 code(s) are NOT |
| | | specified. |
| | | If a CBC consists of two letters, then the only |
| | | unspecified element in the claim triad is the |
| | | diagnosis/ICD-10 code(s). |
| | | There may be situations where this hierarchy does |
| | | not hold true, for example, a diagnosis/ICD-10 code |
| | | is specified, but no CPT/HCPCS codes or fees. In this |
| | | case, the underscore symbol will be used to indicate |
| | | the first two letters are blank: __A |
| E | afg | Optional Claim Specifiers: several health insurance claim |
| | | elements are optional and only used for certain claims. This final |
| | | section of the HICCS is specified with lower case letters to indicate |
| | | that one or more of the following claim elements are needed, to be |
| | | entered by the person or entity filing the claim. Letters a-h will be |
| | | used for this section of the HICCS. Parenthetical numbers indicate |
| | | corresponding data field on the CMS-1500. |
| | | a) Date of Injury or, for pregnancy, Date of Last Menstrual |
| | | Period (14) |
| | | b) Other date (15) |
| | | c) The date at which the patient was unable to work (16) |
| | | d) Referring provider name and NPI number (17) |
| | | e) Dates of hospitalization (18) |
| | | f) Outside Lab (20) |
| | | g) Resubmission code (& Original Ref No) (22) |
| | | h) Prior Authorization Number (23) |
| | | Example: the optional claim specifier “afg” means that the claim |
| | | requires fields 14, 20, and 22 on the CMS-1500 and their |
| | | corresponding data elements. |

The code can be printed in such a manner that permits digital scanning, interpretation, and processing of the health insurance claim data. In one embodiment, each code can be scanned using a QR code or similar rapid-scan implement.

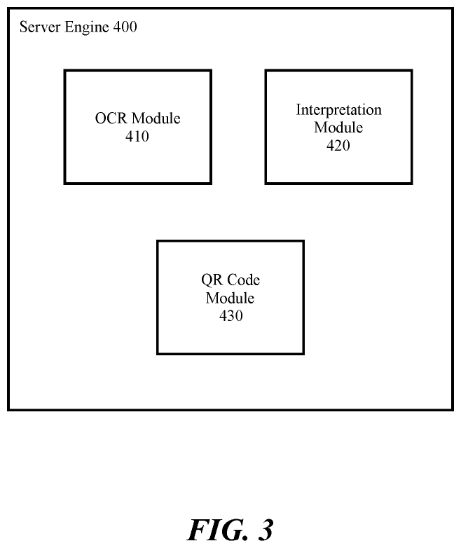

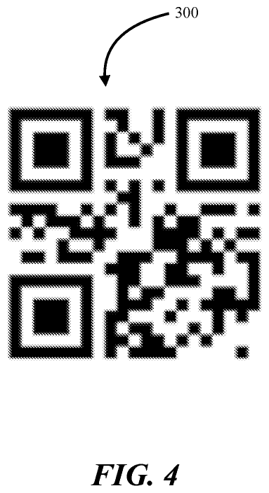

In one embodiment, the code is printed with an “RQ” prefix and printed in the following format: RQ::RLOW987AA6D4C. The presentation of the code shows the RQ:: prefix, which is a unique sequence of characters that do not occur by chance. This sequence permits optical character recognition (OCR) of the code when a receipt is scanned. FIG. 3 illustrates a server engine 400 having an OCR module 410, interpretation module 420, and QR code interpretation module 430 to perform the aforementioned tasks. In one embodiment, the code having format RQ::RLOW987AA6D4C can be presented as the QR code 500 illustrated in FIG. 4. The ability of the code to be interpreted via the interpretation module 420, however executed, permits rapid and error-free processing of medical insurance claims by the system.

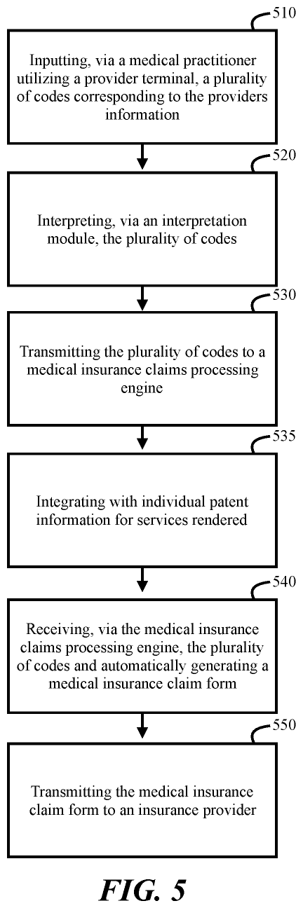

In reference to FIG. 5, a flowchart is provided to illustrates a method for automatically generating a medical insurance claim form. In step 510, the medical practitioner inputs, via the provider terminal, the plurality of codes corresponding to patient information gained from a patient visit. In step 520, the plurality of codes are interpreted via the interpretation module and are transmitted to a medical insurance claims processing engine in step 530. In step 535, individual patent information is integrated for services rendered. In step 540, the medical insurance claims processing engine receives the plurality of codes and automatically generates a medical insurance claim form utilizing the interpreted codes. In step 550, the medical insurance claim form is then transmitted, via the network, to an insurance payer.

In an embodiment, the code utilizes a character to indicate a missing code identifier. In one example, the code RLOW987ACXA having an “X” indicates a missing subsection code or missing fee information. To allow for flexibility, the code generation protocol and structure can be changed.

The following examples refer to code construction references for a provider. In the preferred embodiment, each base code is comprised of a total of eight alphanumeric characters. The eight characters of the base code will be followed by the clinical bundle code. Table 2 illustrates an embodiment wherein the clinical bundle code is comprised of three characters. The resulting codes are transmitted to the CMS-1500 form or 837P providing an accurate and easy to interpret the solution to medical insurance codes.

BEST CITY HOSPITAL

a) Inpatient practice

b) Type II NPI ends in 3233

c) EIN ends in 4344

d) Address: 123 Main St, Knoxville, Tenn.

e) HICCS Base Code: RLOW987A
i) Boxes 25, 32, 33: this organizations information is used in boxes
ii) Box 24B, Place of service code is 21—Inpatient Hospital
iii) Box 27 is marked YES (doctor accepts contracted insurance rates at this practice location)

BEST CITY HOSPITAL—OUTPATIENT CLINIC

a) Outpatient practice

b) Type II NPI ends in 5455

c) EIN ends in 6566

d) Address: 123 Main St, Knoxville, Tenn.

e) HICCS Base Code: RLOW987B
i) Boxes 25, 32, 33: this organizations information is used in boxes
ii) Box 24B, Place of service code is 22—On-Campus Outpatient Hospital
iii) Box 27 is marked YES (doctor accepts contracted insurance rates at this practice location)

Nursing Home Consultants, PLLC (Business Entity)

a) Type II NPI ends in 7677

b) EIN ends in 8788

c) Address: 567 Church St, Knoxville, Tenn.
i) BEST CITY NURSING HOME
   (1) Type II NPI ends in 9009
   (2) Address: 333 Post Rd, Knoxville, Tenn.
   (3) HICCS Base Code: RLOW987C
      (a) Box 32: BEST CITY NURSING HOME is listed
      (b) Boxes 25, 33: Information for Nursing Home Consultants LLC is used
      (c) Box 24B, Place of service code is 31—Skilled Nursing Facility
      (d) Box 27 is marked YES (doctor accepts contracted insurance rates at this practice location)
ii) WORST CITY NURSING HOME
   (1) Type II NPI ends in 7007
   (2) Address: 666 Post Rd, Knoxville, Tenn.
   (3) HICCS Base Code: RLOW987D
      (a) Box 32: WORST CITY NURSING HOME is listed
      (b) Boxes 25, 33: Information for Nursing Home Consultants LLC is used
      (c) Box 24B, Place of service code is 31—Skilled Nursing Facility
      (d) Box 27 is marked YES (doctor accepts contracted insurance rates at this practice location)

Robert Lowry MD, private solo practice

a) Type II NPI—NONE

b) Dr. Lowry's social security number ends in 9899

c) Address: 777 Riverside Drive, Knoxville, Tenn.

d) HICCS Base Code: RLOW987E
i) Boxes 25, 32, 33: this organizations information is used in boxes
ii) Box 24B, Place of service code is 11—Office
iii) Box 27 is marked NO (doctor does NOT accept insurance rates at this practice location)

Table 2 illustrates clinical bundle codes for a health provider. Resulting clinical bundle codes from FIG. 2 include ABB (new evaluation of a patient for hospital admission for diabetes), CCD (new nursing home evaluation for a patient after a concussion), BDC (new outpatient evaluation for gout), and DEA (follow up outpatient visit and EKG, wherein the patient was diagnosed with hypertension).

TABLE 2
Clinical Bundle Codes for a Provider

| | HICCS | | | | |
|---|---|---|---|---|---|
| Code | CBC | CPT | | IDC-10 | |
| Subsection | Identifier | Code(s) | Fee | Code(s) | Notes |
| D1 | A | 99223 | | | |
| | B | 99204 | | | |
| | C | 99304 | | | |
| | D | 99213 | | | |
| | | & | | | |
| | | 93000 | | | |
| D2 | A | | $100 | | |
| | B | | $150 | | |
| | C | | $175 | | |
| | D | | $200 | | |
| | E | | $100 & | | |
| | | | $50 | | |
| D3 | A | | | I10 | |
| | | | | (hypertension) | |
| | B | | | E11.9 | |
| | | | | (diabetes) | |
| | C | | | M10.9 (gout) | |
| | D | | | F07.81 (post- | (these are |
| | | | | concussion | post-accident |
| | | | | syndrome) | evaluations, |
| | | | | | box 10 on |
| | | | | | CMS-1500) |

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombination of the embodiments described herein, and of the manner and process of making and using them, and shall support claims to any such combination or subcombination.

An equivalent substitution of two or more elements can be made for any of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A health insurance claim coding system, comprising:
a network and a database in communication with the network to store patient data and medical practitioner data;
a provider terminal utilized by the medical practitioner to input a plurality of diagnostic codes each corresponding to a patient visit; and
a health insurance claim processing engine to receive the plurality of diagnostic codes and automatically generate a health insurance claim form from at least one of the plurality of codes, the health insurance claim form transmitted via the network to an insurance payer.

2. The system of claim 1, wherein the health insurance claims processing engine receives at least one of the following: an exam name, one or more diagnostic codes, one or more CPT codes, and a fee.

3. The system of claim 2, wherein the fee is associated with the exam name.

4. The system of claim 1, wherein the medical practitioner inputs a plurality of exam names each stored in the database.

5. The system of claim 4, wherein the medical practitioner inputs a plurality of diagnostic codes each corresponding to a patient diagnosis, and wherein the plurality of diagnostic codes are stored in the database.

6. The system of claim 5, wherein each of the plurality of diagnostic codes is customizable via the medical provider.

7. The system of claim 1, wherein the network is accessible by one or more insurance payers.

8. A health insurance claim coding system, comprising:
a network and a database in communication with the network to store patient data and medical practitioner data;
a provider terminal utilized by the medical practitioner to input a plurality of codes each corresponding to a patient visit;
an interpretation module to interpret the plurality of codes input by the medical practitioner; and
a health insurance claim processing engine in communication with the interpretation module, the health insurance claims processing engine receives the plurality of diagnostic codes and automatically generates a health insurance claim form from at least one of the plurality of codes, the health insurance claim form transmitted via the network to an insurance payer.

9. The system of claim 8, wherein the plurality of codes are transmitted to a QR code module to generate a QR code corresponding to the plurality of codes.

10. The system of claim 9, wherein the interpretation module interprets the QR code and transmits the interpreted QR code to the health insurance claim processing engine.

11. The system of claim 9, further comprising an optical character recognition module to interpret the plurality of codes.

12. The system of claim 8, wherein the medical insurance claim processing engine receives at least one of the following: an exam name, one or more diagnostic codes, one or more CPT codes, and a fee.

13. The system of claim 12, wherein the fee is associated with the exam name.

14. The system of claim 8, wherein the medical practitioner inputs a plurality of exam names each stored in the database.

15. The system of claim 14, wherein the medical practitioner inputs a plurality of diagnostic codes each corresponding to a patient diagnosis, and wherein the plurality of diagnostic codes are stored in the database.

16. The system of claim 15, wherein each of the plurality of diagnostic codes is customizable via the medical provider.

17. The system of claim 8, wherein the network is accessible by one or more insurance payers.

18. A method for generating a health insurance claim form, the method comprising the steps of:
inputting, via a medical practitioner utilizing a provider terminal, a plurality of codes corresponding to patient information;
interpreting, via an interpretation module, the plurality of codes;
transmitting the plurality of codes to a health insurance claim processing engine;
receiving, via the health insurance claims processing engine, the interpreted codes and automatically generating a health insurance claim form using the plurality of codes; and
transmitting, via a network, the health insurance claim form to an insurance payer.

19. The method of claim 18, wherein the plurality of codes are transmitted to a QR code module to generate a QR code corresponding to the plurality of codes.

20. The method of claim 19, wherein the interpretation module interprets the QR code and transmits the interpreted QR code to the health insurance claim processing engine.
